(19) United States
(12) Patent Application Publication
(10) Pub. No.: US 2026/0166005 A1
(43) Pub. Date: Jun. 18, 2026

(54) AN ORALLY DISINTEGRATING TABLET CONTAINING ATORVASTATIN AND PROCESS OF PREPARING THE SAME

(71) Applicant: NOVUMGEN LIMITED, London (GB)

(72) Inventor: Kamleshkumar PATEL, London (GB)

(21) Appl. No.: 19/115,053

(22) PCT Filed: Nov. 3, 2023
(86) PCT No.: PCT/GB2023/052871

§ 371 Date: Mar. 25, 2025

(30) Foreign Application Priority Data
Nov. 8, 2022 (GB) 2216584.9

(51) Int. Cl. A61K 31/40; A61K 9/00; A61K 9/20

(52) U.S. Cl. CPC A61K 31/40; A61K 9/0056; A61K 9/2009; A61K 9/2013; A61K 9/2018; A61K 9/2054; A61K 9/2095

(57) ABSTRACT

The present invention relates to an orally disintegrating tablet obtainable by a wet granulation method comprising atorvastatin or pharmaceutically acceptable salts thereof, at least a diluent, a disintegrant agent, a solubilizer, and a stabilizing agent. The orally disintegrating tablet that can disintegrate in the buccal cavity upon contact with saliva in less than 3 minutes preferably less than seconds. The present invention is to provide taste-masking properties and present pleasant palatability such that the administration of the orally disintegrating tablet is not unpleasant for children, elderly, or unconscious patients, and thus patient compliance is improved.

### FIELD OF THE INVENTION

The present invention relates to a pharmaceutical composition of atorvastatin. The present invention relates to an orally disintegrating tablet of atorvastatin or pharmaceutically acceptable salts thereof for oral administration. The present invention also relates to the process of the preparation of the same.

### BACKGROUND OF THE INVENTION

Atorvastatin was first disclosed in the U.S. Pat. No. 5,273,995. Atorvastatin is a lipid-lowering drug included in the statin class of medications. Atorvastatin lowers abnormal cholesterol and lipid levels while increasing high-density lipoprotein-cholesterol concentrations. Atorvastatin reduces the risk of atherosclerosis by reducing apolipoprotein B, low-density lipoprotein cholesterol, and triglyceride plasma concentrations.

Atorvastatin is effective in treating several types of dyslipidemias, including primary hyperlipidemia, mixed dyslipidemia, hypertriglycemia, primary dysbetalipoproteinemia, homozygous familial hypercholesterolemia, heterozygous familial hypercholesterolemia in adolescent patients with failed dietary modifications. Atorvastatin is used as a preventive agent for non-fatal myocardial infarction, fatal and non-fatal stroke, revascularization procedures, hospitalization for congestive heart failure, and angina in patients with coronary heart disease.

Atorvastatin was synthesized in the amorphous form, and most of the studies were conducted on tablets prepared from this material. The amorphous form of atorvastatin is hygroscopic and unstable because it picks up moisture easily and is reactive with atmospheric oxygen, causing the formation of epoxide impurity. Later, an atorvastatin crystalline form with greater stability was developed. The crystalline forms of atorvastatin are Form I, Form II, and Form IV, respectively.

Furthermore, Form I atorvastatin is more stable. Amorphous atorvastatin is much harder to isolate than the crystalline Form of atorvastatin.

The IUPAC name of atorvastatin is (3R,5R)-7-[2-(4-fluorophenyl)-3-phenyl-4-(phenylcarbamoyl)-5-propan-2-ylpyrrol-1-yl]-3,5-dihydroxyheptanoic acid. The plasma half-life of atorvastatin is substantially longer, at 18 to 24 hours. Atorvastatin belongs to the biopharmaceutics classification system (BCS) class II, which has high permeability and low solubility. Drug solubility and drug particle size are intrinsically linked; as a particle gets smaller, the surface area to volume ratio rises. Solubility increases as a result of increased solvent interaction due to the larger surface area.

Atorvastatin is marketed, particularly as an oral tablet and suspension. The commercially marketed products of atorvastatin in tablet form are available in four dosage strengths: 10 mg, 20 mg, 40 mg, and 80 mg for oral administration. In the U.K, the dosage strength for suspension is 4 mg/ml for oral administration. Although atorvastatin is highly efficient when taken orally, its potent bitter taste after oral ingestion makes the development of oral pharmaceutical formulations particularly difficult.

The orally disintegrating tablet of the present invention is a pharmaceutical formulation that rapidly disintegrates in the mouth. The orally disintegrating tablet in the mouth makes it possible, in particular for a patient to be administered atorvastatin without having to simultaneously drink the liquid to ingest the formulation. For patients who have difficulty swallowing, such as children, the elderly, and patients with other conditions, taking atorvastatin in the form of an orally disintegrating tablet may be easier.

U.S. Pat. No. 5,686,104 discloses a tablet and capsule of atorvastatin that comprises at least each diluent, disintegrant, surfactant, lubricant, antioxidant, and stabilizer. The process of this invention involves the milling of the drug while dissolving the binder in an aqueous surfactant solution. The milled drug is then mixed with diluent, alkaline earth metal salt and disintegrant in a rotary mixing vessel. Granulating the blended drug mixture with the surfactant and binder solution in a mixing vessel, then drying the granulated drug mixture overnight at about 50° C. Sieving the dried granulated drug mixture. Tumble blended the sieved drug mixture with the remaining amount of the disintegrant additive and mix it separately with magnesium stearate and then compressed it into tablets.

US 2009/0226515A1 discloses an oral tablet containing atorvastatin and an aminoalkyl methacrylate copolymer dispersed therein, such as dimethyl aminoethyl methacrylate. The process of the invention comprises preparing a granule of atorvastatin and at least one aminoalkyl methacrylate copolymer mixed therewith and compressing the resulting granule into a tablet.

US 2012/0244220A1 discloses a coated tablet of atorvastatin or a pharmaceutically acceptable salt, or solvate thereof, coated with a coating agent. The uncoated tablet comprises a diluent, a disintegrant, and a binder and can be produced by mixing, granulation, drying, particle size regulation, and tableting. Afterward, a polyvinyl alcohol copolymer is used to coat the uncoated tablet. The tablet coating may have an impact on the pharmacodynamic characteristics of drug formulations, and the process can occasionally result in coating defects such as chipping and cracking. As a result, the procedure needs to be carried out by highly skilled experts.

EP 3184103A1 discloses a compressed tablet core of atorvastatin in the form of pellets or the form of granules. The tablet is prepared by the direct compression method, which includes the steps of mixing and sieving of atorvastatin or a pharmaceutically acceptable salt thereof with a filler, a stabilizing additive, a glidant, a binder, and a disintegrant. The process involves blending the mass; lubricating the blend with a lubricant; and compressing the blend to form tablet cores, which further comprise a coating of hydroxypropyl methylcellulose.

US 2019/0209526A1 discloses an oral atorvastatin suspension composition in which solid atorvastatin particles are suspended in the carrier. The process involves mixing atorvastatin with at least one non-polar carrier. Preferably, the non-polar carrier is miglyol 812. Additional components, such as sweeteners, flavoring agents, and thickening agents are added to the mixture. The mixing continues until the atorvastatin is homogeneously dispersed in the non-polar carrier. Pharmaceutical suspensions are unstable, and for this reason, it requires formulation skills to ensure that the physical stability of the formulation is retained over the period of the shelf-life.

US 2013/0137745A1 discloses a parenteral liquid pharmaceutical formulation of atorvastatin with a pharmaceutically acceptable complexing agent, which is cyclodextrin. The process of the invention comprises preparing lyophilized particles by adding atorvastatin to a mixture of a complexing agent and a suitable solvent. The mixture is vortexed and sonicated and the pH of the mixture is adjusted using a buffer and lyophilizing the mixture to obtain lyophilized particles. Reconstituted the lyophilized particles in a pharmaceutically acceptable solution for injection. Injectable formulations have several issues, including the possibility of incorrect dosing and adverse effects, stability issues, the risk of blood clot formation, and so on. Furthermore, trained nursing staff or a trained individual is required to administer the medication.

The orally disintegrating tablet can be administered to any patient particularly children and the elderly, who have trouble swallowing the tablets, even with liquids. In contrast, the liquid dosage form is bulky, difficult to transport, and takes up a lot of space. Due to their inherent instability, liquid dosage forms often have shorter shelf lives. The patient's measurement of the exact volume determines whether the dose is administered appropriately, which increases the chance for variability. It is therefore highly desirable to remedy this issue and create an orally disintegrating tablet containing atorvastatin that has taste-masking properties, has pleasant palatability, and ensures patient comfort during administration.

### SUMMARY OF THE INVENTION

Accordingly, the present invention provides an orally disintegrating tablet that comprises atorvastatin or pharmaceutically acceptable salts thereof, as well as taste-masking properties, and presents pleasant palatability such that the administration of the orally disintegrating tablet is not unpleasant for children, the elderly, or the unconscious patients.

Another embodiment of the present invention, the orally disintegrating tablet for oral administration is prepared. The orally disintegrating tablet comprises atorvastatin or pharmaceutically acceptable salts thereof, in addition to the stabilizing additive calcium carbonate, at least one other ingredient such as a binder, diluent, disintegrant, lubricant, and solubilizer.

Further, another embodiment of the present invention involves a process for preparing the orally disintegrating tablet for oral administration. The orally disintegrating tablet is prepared by the wet granulation method.

Another embodiment of the present invention can effectively treat angina, hyperlipidemia, atherosclerosis, dyslipidemia, hypercholesterolemia, hypertriglyceridemia, dysbetalipoproteinemia, myocardial infarction, and stroke.

### OBJECTS OF THE INVENTION

The primary object of the present invention is to provide an orally disintegrating tablet of atorvastatin or pharmaceutically acceptable salts thereof.

It is an object of the present invention to enhance the disintegration and dissolution of the drug for oral administration.

It is a further another object of the present invention is to provide a process for preparing the orally disintegrating tablet of atorvastatin pharmaceutically acceptable salts thereof, for oral administration.

It is a further another object of the present invention is to provide a stable orally disintegrating tablet of atorvastatin or pharmaceutically acceptable salts thereof.

It is a further another object of the present invention is to provide taste-masking properties and present pleasant palatability such that the administration of the orally disintegrating tablet is not unpleasant for children, the elderly, or unconscious patients.

Yet another object of the present invention is to provide fast disintegration of the dosage form as it gets in contact with saliva with a good agreeable mouth feeling.

### DETAILED DESCRIPTION OF THE INVENTION

The present invention relates to an orally disintegrating tablet comprising atorvastatin or a pharmaceutically acceptable salt thereof, suitable for oral administration. It also relates to its preparation method and the use of the orally disintegrating tablet. The term “orally disintegrating tablet” means a tablet that can disintegrate in the oral cavity in less than 3 minutes.

Despite tremendous innovations in drug delivery, the oral route remains the preferred route for the administration of therapeutic agents because of accurate dosage, low-cost therapy, self-medication, non-invasive method, and ease of administration, leading to a high level of patient compliance. However, some geriatric patients may find it difficult to take conventional tablets and capsules administered with a glass of water due to changes in various physiological and neurological conditions associated with ageing, such as difficulty swallowing, dysphagia, hand tremors, deterioration in their eyesight, hearing, memory, risk of choking, in addition, to change in taste and smell. Solid dosage forms also present significant administrative challenges in other patient groups, such as children, the mentally challenged, bedridden, and uncooperative patients. Moreover, patients traveling with little or no access to water limit the utility of orally administered conventional tablets or capsules. Therefore, to cater to the needs of such patients, recent advancements in technology have resulted in the development of viable dosage alternatives, popularly known as orally disintegrating tablets (ODTs). These dose forms are preferred alternatives for oral medication in terms of improving patient acceptability and quality of life.

Orally disintegrating tablets are also known as rapidly disintegrating tablets, orodispersible tablets, or orally dispersible tablets. Orally disintegrating tablets are solid unit dosage forms that contain drugs that disintegrate rapidly in the oral cavity, usually in a matter of seconds even without the use of water. Through pregastric absorption from the mouth, pharynx, and esophagus, the tablet's disintegration in the mouth can improve the drug's therapeutic efficacy. When compared to conventional tablets, avoiding first-pass hepatic metabolism significantly increases drug bioavailability.

Drug absorption affects a drug's bioavailability. In the case of poorly water-soluble drugs, such as atorvastatin, the bioavailability of a drug is mostly dependent on its dissolution, which is influenced by drug dosage from disintegration. Poorly soluble drug dissolution rates are closely correlated with the particle size distribution. Tablets prepared using atorvastatin and having a range of particle sizes have been found to have the desired disintegration properties. In one embodiment, the atorvastatin has a D 10 particle size distribution of from about 0.25 to 15 μm, more preferably in the range of 0.5 to 10 μm. In a further embodiment, the atorvastatin has a D 50 particle size distribution of from about 1 to 20 μm, more preferably in the range of 4 to 10 μm. In a more preferred embodiment, the atorvastatin has a D 90 particle size distribution of from about 20 to 200 μm, more preferably in the range of 50 to 150 μm. As a result, an orally disintegrating tablet formulation could aid in drug dissolution and absorption.

According to one embodiment of the present invention is an orally disintegrating tablet suitable for oral administration comprising atorvastatin, at least one disintegrant, and at least one diluent. When in contact with saliva, this tablet disintegrates, releasing the active ingredient and ensuring maximum drug bioavailability compared to conventional dose forms. In one embodiment, the atorvastatin calcium trihydrate is present in the range of about 5% w/w to about 15% w/w, preferably in the range of about 8% w/w to about 12% w/w and is administered as an oral solid dosage form.

The term “about”, as and when used in this specification, means ±10% of the mentioned value.

The orally disintegrating tablet prevents the first-pass effect of hepatic enzymes right after absorption and prevents drugs from coming into contact with digestive enzymes in the GI tract. The direct access to blood circulation, and the avoidance of any drug metabolism, results in quickly achieving the maximum levels of the active ingredient in the plasma. Thus, a faster onset of pharmacological effects of the drug in patients is achieved in comparison to conventional oral delivery where the composition is swallowed.

As per one embodiment, the orally disintegrating tablet of the present invention comprises atorvastatin or pharmaceutically acceptable salts thereof, disintegrant and diluent. In addition, the orally disintegrating tablet further comprises at least one pharmaceutically acceptable excipient selected from a sweetener, flavoring agent, binder, stabilizing agent, solubilizer, and lubricant. The present invention has been made to solve the above-mentioned problems, and its object is to use atorvastatin or a pharmaceutically acceptable salt thereof as an active ingredient, which simultaneously prevents not only bitterness but also poor dissolution.

As per one embodiment of the present invention, the composition comprises a diluent selected from dextrates, microcrystalline cellulose, dextrose, fructose, Sorbitol, pregelatinized starch, starch, xylitol, sucrose, maltodextrin, maltose, mannitol and combinations thereof. In the present invention combination of the microcrystalline cellulose and mannitol is preferred as a diluent in the range of about 10% w/w to about 80% w/w, preferably in the range from about 15% w/w to about 65% w/w. microcrystalline cellulose is present in the range from about 10% w/w to about 60% w/w, preferably from about 15% w/w to about 40% w/w, and mannitol is present in the range from about 30% w/w to about 85% w/w, preferably from about 45% w/w to about 70% w/w. In the development of tablets, the use of mannitol as a filler has increased due to its physicochemical properties, such as its lower hygroscopicity, chemical inertness, and advantageous tableting behavior, including compactability. Lactose contains water in some amounts, which can affect the stability of API. Mannitol provides patients with a pleasant, cool feeling in the mouth. Furthermore, mannitol is more chemically inert than lactose because mannitol is not accompanied by the Maillard reaction, which can occur between lactose and amine functional groups in APIs, which leads to their degradation. Thus, it can be used as an alternative filler to lactose. Microcrystalline cellulose is one of the types of fillers that has swelling tendencies and excellent water imbibing or wicking action. When employed as a filler in the wet granulation process, microcrystalline cellulose's wicking action encourages rapid wetting of the powder mixture, lowers the wet mass's sensitivity to over-wetting, and speeds up the drying process. By using microcrystalline cellulose, faster disintegration of the tablet will be obtained. Therefore, it is used in combination with microcrystalline cellulose in comparison with other diluents in the present invention.

As per one more embodiment of the present invention, the composition comprises the disintegrant selected from methylcellulose, alginic acid, carboxymethylcellulose calcium, guar gum, carboxymethylcellulose Sodium, polacrilin potassium, croscarmellose sodium, poloxamer, sodium starch glycolate, and sodium alginate. Further, the disintegrant can be single or any combination thereof. Croscarmellose sodium is the preferred disintegrant for the present invention in the range from about 0.25% w/w to about 8% w/w, preferably in the range from about 0.5% w/w to about 6.5% w/w. Croscarmellose sodium accelerates the wicking process, through the mechanisms of swelling, recovering elastic energy, and capillary action, ultimately reducing the disintegration time. Additionally, it has a tendency to absorb water quickly, which causes significant swelling. Therefore, this rapid absorption of water by croscarmellose sodium results in a significant increase in the volume of granules, resulting in rapid and uniform disintegration. As a result, the preferred disintegrant for an orally disintegrating tablet containing atorvastatin calcium is croscarmellose sodium.

As per another embodiment of the present invention, at least one further pharmaceutically acceptable excipient is a lubricant selected from boric acid, magnesium stearate, Sodium Stearyl fumarate, micronized polyoxy ethylene glycol, leucine, sodium benzoate, sodium acetate, sodium lauryl sulfate, stearic acid, sodium stearate, sodium oleate, calcium stearate, waxes or mixtures thereof. Magnesium stearate is preferred as a lubricant for the present invention and is present in the range from about 0.25% w/w to about 6% w/w, preferably in the range from about 0.5% w/w to about 4% w/w.

As per another embodiment of the present invention, at least one further pharmaceutically acceptable excipient is a sweetener selected from cyclamate, acesulfame potassium, neo hesperidin dihydrochalcone, monoammonium glycyrrhizinate, saccharin sodium, sucralose, saccharin, aspartame or mixtures thereof. Aspartame is preferred as a sweetener for the present invention and is present in the range of about 0.25% w/w to about 7.5% w/w, preferably in the range of about 0.5% w/w to about 4% w/w.

As per one another embodiment of the present invention, the composition comprises a flavoring agent, for example, menthol, floral fennel flavor, mint powder, vanillin, or orange flavor. The orally disintegrating tablet of the present invention can be prepared in the presence of orange flavor present in the range from about 0.25% w/w to about 5% w/w, preferably in the range from about 0.5% w/w to about 4% w/w.

As per another embodiment of the present invention, the orally disintegrating tablet further comprises a stabilizing agent selected from calcium carbonate, calcium hydroxide, magnesium carbonate, magnesium hydroxide, magnesium silicate, magnesium aluminate, or aluminum magnesium hydroxide and their combinations thereof. The orally disintegrating tablet of the present invention can be prepared in the presence of calcium carbonate in the range of about 2% w/w to about 15% w/w, preferably in the range of about 4% w/w to about 9% w/w.

As per one embodiment, a suitable binder for the present invention can be selected from the group consisting of alginic acid, carbomer, ethyl cellulose, gelatine, glucose, guar gum, hydroxy ethyl cellulose, hydroxypropyl cellulose, hydroxypropyl methylcellulose, methylcellulose, polydextrose, polyethylene oxide, and Povidone K30. Preferably, hydroxypropyl cellulose is preferred as a binder for the present invention. It is present in the range from about 0.25% w/w to about 10% w/w, preferably in the range from about 0.5% w/w to about 8.5% w/w.

As per one another embodiment, suitable solubilizer for present invention can be selected from the group consisting of heptadecaethylene oxycetanol, lecithins, sorbitol monooleate, polyoxyethylene sorbitol monooleate, polyoxyethylene stearate, polyoxyethylene sorbitan monolaurate, benzalkonium chloride, nonoxynol 10, oxtoxynol 9, polysorbates 20, 40, 60 or 80, sorbitan monoalmitate, sodium salts of fatty alcohol-sulfates such as sodium lauryl sulfate, sodium dodecyl sulfate, sodium salts of sulfosuccinates, such as sodium dioctyl sulfosuccinate, partially esters of fatty acids with alcohols such as glycerine monostearate, partially esters of fatty acids with sorbitans such as sorbitan monolaurate, partially esters of fatty acids with polyhydroxyethylene sorbitans such as polyethyleneglycol sorbitan monolaurate, monostearate or monooleate, ethers of fatty alcohols with polyhydroxyethylene, esters of fatty acids with polyhydroxyethylene, copolymers of ethylene oxide and propylene oxide and ethoxylated triglycerides. Polysorbate 80 is preferred as a solubilizer for the present invention and is present in the range from about 0.02% w/w to about 6% w/w, preferably in the range from about 0.05% w/w to about 4% w/w.

In the preferred embodiment of the present invention is to provide an orally disintegrating tablet suitable for oral administration comprising atorvastatin or pharmaceutically acceptable salts thereof, at least one disintegrant, and at least one diluent. The orally disintegrating tablet further comprises at least one pharmaceutically acceptable excipient selected from a sweetener, flavoring agent, binder, solubilizer, stabilizing agent, and lubricant.

Another embodiment of the present invention is the use of the wet granulation process for the preparation of orally disintegrating tablets containing atorvastatin or salts thereof, which is one of the most economical methods. The wet granulation method is primarily used to improve the flow and compressibility of powders as well as to prevent the segregation of the blend's components. The wet granulation method was preferred over other conventional manufacturing processes because it improves the tablets' hardness by reducing friability.

As per one another embodiment, the disintegrating time of the atorvastatin orally disintegrating tablet is not more than 3 minutes, preferably less than 90 seconds.

As per another embodiment, 80% of the atorvastatin is released in 60 minutes, preferably more than 90% is released within 50 minutes.

In one preferred embodiment, the wet granulation method is used to prepare the orally disintegrating tablet. Atorvastatin calcium, microcrystalline cellulose 101, mannitol, croscarmellose sodium, calcium carbonate, and hydroxypropyl cellulose are sieved separately through a 40# sieve. Aspartame, Orange flavor, and magnesium stearate are Sieved separately through a 60# sieve. The binder solution was prepared by adding one half quantity of hydroxy propyl cellulose and polysorbate 80 to a sufficient quantity of purified water. The previously sifted atorvastatin calcium and microcrystalline cellulose 101 were loaded into the rapid mixer granulator and mixed for 10 minutes by keeping the impeller slow and the chopper off. The binder solution was slowly added to the dry mix with continuous mixing in a rapid mixer granulator for 5 min by keeping the impeller slow and the chopper off and then granulating the mixture for a further 2 min. The Granulated blend is then dried in a dryer at 50° C.±5° C. till the loss on drying of the dried granules becomes not more than 2.5%. Dried granules were sifted through a 24# sieve. To the dried granules, the mannitol, remaining quantity of hydroxyl propyl cellulose, croscarmellose sodium, calcium carbonate, aspartame, and orange flavor were added and blended for 15 minutes in a blender, and then magnesium stearate was added to the pre-lubricated blend and mixed for 5 minutes.

The invention is further illustrated by the following examples, which are by no means intended to limit the scope of the invention but are given by way of illustration.

### Example 1

The Orally disintegrating tablet was made according to the method defined below using the formulation having the ingredients shown in table I:

TABLE I

| Ingredients | Quantity (mg/tablet) | % w/w |
|---|---|---|
| Atorvastatin | 21.690 | 10.845 |
| Microcrystalline cellulose pH-102 | 40.00 | 20.000 |
| HPC | 6.00 | 3.00 |
| Mannitol | 107.31 | 54.50 |
| Croscarmellose sodium | 4.00 | 2.00 |
| Calcium Carbonate | 14.00 | 7.00 |
| Aspartame | 2.50 | 1.25 |
| Orange flavor | 1.50 | 0.75 |
| Magnesium stearate | 3.00 | 1.50 |
| Total | 200.00 | 100.00 |

### Manufacturing Process:

Atorvastatin calcium is mixed with approximately 1/10 quantity of microcrystalline cellulose. The prepared mixture is sieved through a 40# sieve. Mannitol, microcrystalline cellulose-102, Hydroxypropyl Cellulose, Croscarmellose Sodium, and Calcium carbonate are sieved separately through a 40# sieve. Aspartame, orange flavor, and magnesium stearate are sieved separately through a 60# sieve. Mannitol, microcrystalline cellulose 102, Croscarmellose Sodium, orange flavor aspartame, atorvastatin calcium, and calcium carbonate are blended in a blender and mixed for 20 minutes and then magnesium stearate is added to the final mixture and properly mixed for 5 minutes.

The prepared blended mixture shows the characteristics mentioned in the table below:

| Test Parameters of blend | Result |
|---|---|
| Bulk Density (gm/ml) | 0.520 |
| Tap Density (gm/ml) | 0.823 |
| Carr's Index (%) | 36.84% |
| Hausner's Ratio | 1.58 |

### Example 2

The orally disintegrating tablet was prepared according to the procedure described below using the formulation having the ingredients shown in table II:

TABLE II

| Ingredients | Quantity (mg/tablet) | % w/w |
|---|---|---|
| Atorvastatin | 21.690 | 10.845 |
| Microcrystalline Cellulose 101 | 40.00 | 20.000 |
| HPC | 6.00 | 3.00 |
| Water | Q.s. | Q.s. |
| Mannitol | 107.31 | 54.5 |
| Croscarmellose Sodium | 4.00 | 2.00 |
| Calcium Carbonate | 14.00 | 7.00 |
| Aspartame | 2.50 | 1.25 |
| Orange flavor | 1.50 | 0.75 |
| Magnesium Stearate | 3.00 | 1.50 |
| Total | 200.00 | 100.00 |

### Manufacturing Process:

Atorvastatin calcium, microcrystalline cellulose 101, mannitol, croscarmellose sodium, and calcium carbonate are sieved separately through a 40# sieve. Aspartame, Orange flavor, and magnesium stearate are sieved separately through a 60# sieve. The binder solution was prepared by adding hydroxypropyl cellulose to a sufficient quantity of purified water. The sifted atorvastatin calcium and microcrystalline cellulose 101 were loaded into the rapid mixer granulator and mixed for 10 minutes by keeping the impeller slow and the chopper off. The binder solution was slowly added to the dry mix with continuous mixing in a rapid mixer granulator for 5 min by keeping the impeller slow and the chopper off and then granulating the mixture for a further 2 min. The Granulated blend is then dried in a dryer at 50° C.±5° C. till the loss on drying of the dried granules becomes not more than 2.5%. Dried granules were sifted through a 24# sieve. To the dried granules, the mannitol, croscarmellose sodium, calcium carbonate, aspartame, and orange flavor were added and blended for 15 minutes in a blender, and then magnesium stearate was added to the pre-lubricated blend and mixed for 5 minutes.

| Test Parameters of blend | Result |
|---|---|
| Bulk Density (gm/ml) | 0.526 |
| Tap Density (gm/ml) | 0.659 |
| Carr's Index (%) | 20.09 |
| Hausner's Ratio | 1.25 |

The tablets present the characteristics mentioned in the table below:

| Test Parameters | Result |
|---|---|
| Weight (mg) | 200 |
| Hardness (N) | 70 |
| Friability (%) | 0.03 |
| Buccal disintegration time (seconds) | 210 sec |

### Example 3

The orally disintegrating tablet was prepared according to the procedure described below using the formulation having the ingredients shown in table III:

TABLE-III

| Ingredients | Quantity (mg/tablet) | % w/w |
|---|---|---|
| Atorvastatin | 21.690 | 10.84 |
| Microcrystalline Cellulose 101 | 40.00 | 20.000 |
| HPC | 4.00 | 2.00 |
| Water | Q.s. | Q.s. |
| Mannitol | 107.31 | 53.65 |
| Croscarmellose Sodium | 6.00 | 3.00 |
| Calcium Carbonate | 14.00 | 7.00 |
| Aspartame | 2.50 | 1.25 |
| Orange flavor | 1.50 | 0.75 |
| Magnesium Stearate | 3.00 | 1.50 |
| Total | 200.00 | 100.00 |

### Manufacturing Process:

Atorvastatin calcium, microcrystalline cellulose 101, mannitol, croscarmellose sodium, and calcium carbonate are sieved separately through a 40# sieve. Aspartame, Orange flavor, and magnesium stearate are sieved separately through a 60# sieve. The binder solution was prepared by adding hydroxypropyl cellulose to a sufficient quantity of purified water. The sifted atorvastatin calcium and microcrystalline cellulose 101 were loaded into the rapid mixer granulator and mixed for 10 minutes by keeping the impeller slow and the chopper off. The binder solution was slowly added to the dry mix with continuous mixing in a rapid mixer granulator for 5 min by keeping the impeller slow and the chopper off and then granulating the mixture for a further 2 min. The Granulated blend is then dried in a dryer at 50° C.±5° C. till the loss on drying of the dried granules becomes not more than 2.5%. Dried granules were sifted through a 24# sieve. To the dried granules, the mannitol, croscarmellose sodium, calcium carbonate, aspartame, and orange flavor were added and blended for 15 minutes in a blender, and then magnesium stearate was added to the pre-lubricated blend and mixed for 5 minutes.

| Test Parameters of blend | Result |
|---|---|
| Bulk Density (gm/ml) | 0.750 |
| Tap Density (gm/ml) | 0.882 |
| Carr's Index (%) | 15.0% |
| Hausner's Ratio | 1.18 |

The tablets present the characteristics mentioned in the table below:

| Test Parameters | Result |
|---|---|
| Weight (mg) | 200 |
| Hardness (N) | 60 |
| Friability (%) | 0.03 |
| Buccal disintegration time (seconds) | 50 sec |

### Example 4

The orally disintegrating tablet was prepared according to the procedure described below using the formulation having the ingredients shown in table IV:

TABLE IV

| Ingredients | Quantity (mg/tablet) | % w/w |
|---|---|---|
| Atorvastatin | 21.690 | 10.845 |
| Microcrystalline Cellulose 101 | 40.00 | 20.000 |
| HPC | 4.00 | 2.00 |
| Polysorbate 80 | 0.20 | 0.1 |
| Water | Q.s. | Q.s. |
| Mannitol | 107.11 | 53.55 |
| Croscarmellose Sodium | 6.00 | 3.00 |
| Calcium Carbonate | 14.00 | 7.00 |
| Aspartame | 2.50 | 1.25 |
| Orange flavor | 1.50 | 0.75 |
| Magnesium Stearate | 3.00 | 1.50 |
| Total | 200.00 | 100.00 |

### Manufacturing Process:

Atorvastatin calcium, microcrystalline cellulose 101, mannitol, croscarmellose sodium, and calcium carbonate are sieved separately through a 40# sieve. Aspartame, Orange flavor, and magnesium stearate are Sieved separately through a 60# sieve. The binder solution was prepared by adding hydroxy propyl cellulose and polysorbate 80 to a sufficient quantity of purified water. The sifted atorvastatin calcium and microcrystalline cellulose 101 were loaded into the Rapid Mixer Granulator and mixed for 10 minutes by keeping the impeller slow and the chopper off. The binder solution was slowly added to the dry mix with continuous mixing in a rapid mixer granulator for 5 min by keeping the impeller slow and the chopper off and then granulating the mixture for a further 2 min. The Granulated blend is then dried in a dryer at 50° C.±5° C. till the loss on drying of the dried granules becomes not more than 2.5%. Dried granules were sifted through a 24# sieve. To the dried granules, the mannitol, croscarmellose sodium, calcium carbonate, aspartame, and orange flavor were added and blended for 15 minutes in a blender, and then magnesium stearate was added to the pre-lubricated blend and mixed for 5 minutes.

| Test Parameters of blend | Result |
|---|---|
| Bulk Density (gm/ml) | 0.520 |
| Tap Density (gm/ml) | 0.618 |
| Carr's Index (%) | 15.79 |
| Hausner's Ratio | 1.19 |

The tablets present the characteristics mentioned in the table below:

| Test Parameters | Result |
|---|---|
| Weight (mg) | 200 |
| Hardness (N) | 54 |
| Friability (%) | 0.16 |
| Buccal disintegration time (seconds) | 24 sec |

### Example 5

The orally disintegrating tablet was prepared according to the procedure described below using the formulation having the ingredients shown in table V:

TABLE V

| | Quantity (10 | Quantity (20 | |
|---|---|---|---|
| Ingredients | mg/tablet) | mg/tablet) | % w/w |
| Atorvastatin | 10.845 | 21.690 | 10.845 |
| Microcrystalline Cellulose 101 | 20.00 | 40.00 | 20.000 |
| HPC | 4.5 | 9.00 | 4.5 |
| Polysorbate 80 | 0.10 | 0.20 | 0.10 |
| Water | Q.s. | Q.s. | Q.s. |
| Mannitol | 51.90 | 102.11 | 51.05 |
| Croscarmellose Sodium | 3.00 | 6.00 | 3.00 |
| Calcium Carbonate | 7.00 | 14.00 | 7.00 |
| Aspartame | 1.25 | 2.50 | 1.25 |
| Orange flavor | 0.75 | 1.50 | 0.75 |
| Magnesium Stearate | 1.50 | 3.00 | 1.50 |
| Total | 100.00 | 200.00 | 100.00 |

### Manufacturing Process:

Atorvastatin calcium, microcrystalline cellulose 101, mannitol, croscarmellose sodium, calcium carbonate, and hydroxypropyl cellulose are sieved separately through a 40# sieve. Aspartame, Orange flavor, and magnesium stearate are Sieved separately through a 60# sieve. The binder solution was prepared by adding polysorbate 80 and one half quantity of hydroxy propyl cellulose to a sufficient quantity of purified water. The previously sifted atorvastatin calcium and microcrystalline cellulose 101 were loaded into the Rapid Mixer Granulator and mixed for 10 minutes by keeping the impeller slow and the chopper off. The binder solution was slowly added to the dry mix with continuous mixing in a rapid mixer granulator for 5 min by keeping the impeller slow and the chopper off and then granulating the mixture for a further 2 min. The Granulated blend is then dried in a dryer at 50° C.±5° C. till the loss on drying of the dried granules becomes not more than 2.5%. Dried granules were sifted through a 24# sieve. To the dried granules, the mannitol, remaining quantity of hydroxyl propyl cellulose, croscarmellose sodium, calcium carbonate, aspartame, and orange flavor were added and blended for 15 minutes in a blender, and then magnesium stearate was added to the pre-lubricated blend and mixed for 5 minutes.

| Test Parameters | Result |
|---|---|
| Bulk Density (gm/ml) | 0.520 |
| Tap Density (gm/ml) | 0.618 |
| Carr Index (%) | 15.79 |
| Hausner Ratio | 1.19 |

### Example 6: The Dissolution Profile of the Tablet Prepared According to Example 5

A dissolution profile is made with the orally disintegrating tablet of example 5

The conditions of dissolution are the following:
Apparatus: USP type II (paddle)
Rate of rotation: 75 rpm
Volume: 900 ml
Temperature: 37° C.
Detection: Direct UV spectrophotometry at 248 nm
Dissolution medium: at pH 6.8 Phosphate buffer

### Dissolution Profile of Atorvastatin 10 mg Orally Disintegrating Tablet

Example 5 (Cumulative % Drug Release)

pH 6.8 Phosphate buffer

| Time (min) | % Release | % Relative Standard Deviation |
|---|---|---|
| 10 | 82.9 | 4.3 |
| 15 | 88.1 | 4.3 |
| 20 | 90.8 | 4.8 |
| 30 | 93.8 | 1.3 |
| 45 | 97.0 | 1.7 |
| 60 | 98.5 | 1.7 |

### Dissolution Profile of Atorvastatin 20 mg Orally Disintegrating Tablet

Example 5 (Cumulative % Drug Release)

pH 6.8 Phosphate buffer

| Time (min) | % Release | % Relative Standard Deviation |
|---|---|---|
| 10 | 72.8 | 2.1 |
| 15 | 79.2 | 3.0 |
| 20 | 82.6 | 1.7 |
| 30 | 87.3 | 1.0 |
| 45 | 92.0 | 1.1 |
| 60 | 95.5 | 2.3 |

### Example 7

The tablet prepared according to example 5 were subjected to a stability study of 40° C.±2° C./75% RH±5% RH for 1 month. Results are tabulated below.

### Atorvastatin 10 mg Orally Disintegrating Tablets

| | | | | Exp 5 | |
|---|---|---|---|---|---|
| | | | | (After 1 | |
| | | Exp 5 | | month at | |
| Test | Specification | (Initial) | | 40° C. ± 2° C.) | |
| Average weight | 100.0 mg ± 7.5% | 100.1 | mg | 100.0 | mg |
| | (92.5 mg-107.5 mg) | | | | |
| Hardness | 25-75N | 50N | | 54N | |
| Thickness | 2.10 ± 0.30 mm | 2.14 | mm | 2.20 | mm |
| | (1.80 mm to 2.40 mm) | | | | |
| Friability | NMT 1.0% | 0.19% | | 0.20% | |
| Disintegration | NMT 3 Minute | 33 | Sec | 46 | Sec |

| time | | | |
|---|---|---|---|
| Assay | 95.0%-105.0% of the | 99.4 | 98.7 |
| | labelled amount of | | |
| | Atorvastatin calcium | | |
| | trihydrate. | | |
| Dissolution | NLT 75% (Q) labelled | 90.2 | 80.2 |
| | amount of Atorvastatin | | |
| | should be dissolved in | | |
| | 45 minutes. | | |

### Atorvastatin 20 mg Orally Disintegrating Tablets

| | | | | Exp 5 | |
|---|---|---|---|---|---|
| | | | | (After 1 | |
| | | Exp 5 | | month at | |
| Test | Specification | (Initial) | | 40° C. ± 2° C.) | |
| Average weight | 200.0 mg ± 7.5% | 200.2 | mg | 200.1 | mg |
| | (185.0 mg-215.0 mg) | | | | |
| Hardness | 25-75N | 53.7N | | 60N | |
| Thickness | 2.60 ± 0.30 mm | 2.60 | mm | 2.51 | mm |
| | (2.30 mm to 2.90 mm) | | | | |
| Friability | NMT 1.0% | 0.16% | | 0.18% | |
| Disintegration | NMT 3 Minute | 24 | Sec | 49 | Sec |

| time | | | |
|---|---|---|---|
| Assay | 95.0%-105.0% of the | 100.3 | 102.2 |
| | labelled amount of | | |
| | Atorvastatin. calcium | | |
| | trihydrate. | | |
| Dissolution | NLT 75% (Q) labelled | 92.0 | 91.8 |
| | amount of Atorvastatin | | |
| | should be dissolved in | | |
| | 45 minutes. | | |

1. An orally disintegrating tablet of atorvastatin for oral administration comprising;
a) atorvastatin or pharmaceutically acceptable salts thereof;
b) a diluent is a combination of microcrystalline cellulose 101 and mannitol present in the range of about 10% w/w to about 80% w/w, preferably in the range of about 15% w/w to about 65% w/w; and
c) at least one disintegrant.

2. The orally disintegrating tablet according to claim 1, wherein mannitol is present in the range from about 30% w/w to about 85% w/w, preferably from about 45% w/w to about 70% w/w.

3. The orally disintegrating tablet according to claim 1, wherein microcrystalline cellulose 101 is present in the range from about 10% w/w to about 60% w/w, preferably from about 15% w/w to about 40% w/w.

4. The orally disintegrating tablet according to claim 1, wherein the atorvastatin is in a crystalline form I.

5. The orally disintegrating tablet according to claim 4, wherein the therapeutically effective amount of atorvastatin or pharmaceutically acceptable salts thereof, is present in an amount ranging from about 5% w/w to about 15% w/w, preferably in the range from about 8% w/w to about 12% w/w.

6. The orally disintegrating tablet according to claim 1, wherein the pharmaceutical composition further comprises one or more pharmaceutically acceptable excipients, selected from the binder, stabilizing agent, solubilizer, sweetener, flavoring agent, or lubricant.

7. The orally disintegrating tablet according to claim 1, wherein the disintegrant is selected from methylcellulose, alginic acid, carboxymethylcellulose calcium, carboxymethylcellulose sodium, polacrilin potassium, croscarmellose sodium, guar gum, poloxamer, sodium starch glycolate, and sodium alginate or any combination thereof.

8. The orally disintegrating tablet according to claim 7, wherein the disintegrant is Croscarmellose sodium present in the range of about 0.25% w/w to about 8% w/w, preferably in the range of about 0.5% w/w to about 6.5% w/w.

9. The orally disintegrating tablet according to claim 6, wherein the lubricant is selected from the group consisting of boric acid, magnesium stearate, sodium Stearyl fumarate, micronized polyoxyethylene glycol, leucine, sodium benzoate, sodium acetate, sodium lauryl sulfate, stearic acid, sodium stearate, sodium oleate, calcium stearate, and combinations thereof.

10. The orally disintegrating tablet according to claim 9, wherein the lubricant is Magnesium stearate present in the range from about 0.25% w/w to about 6% w/w, preferably in the range from about 0.5% w/w to about 4% w/w.

11. The orally disintegrating tablet according to claim 6, wherein the binder is selected from the group consisting of alginic acid, carbomer, ethyl cellulose, gelatine, glucose, guar gum, hydroxy ethyl cellulose, hydroxypropyl cellulose, hydroxypropyl methylcellulose, methylcellulose, polydextrose, polyethylene oxide, and Povidone K30 present in the range from about 0.25% w/w to about 10% w/w, preferably in the range from about 0.5% w/w to about 8.5% w/w.

12. The orally disintegrating tablet according to claim 6, wherein the solubilizer is selected from the group consisting of heptadecaethylene oxycetanol, lecithins, sorbitol monooleate, polyoxyethylene sorbitol monooleate, polyoxyethylene stearate, polyoxyethylene sorbitan monolaurate, benzalkonium chloride, nonoxynol 10, nonoxynol 9, polysorbates 20, 40, 60 or 80, sorbitan monoacetate, sodium salts of fatty alcohol-sulfates such as sodium lauryl sulfate, sodium dodecyl sulfate, sodium salts of sulfosuccinates, such as sodium dioctyl sulfosuccinate, partially esters of fatty acids with alcohols such as glycerine monostearate, partially esters of fatty acids with sorbitans such as sorbitan monolaurate, partially esters of fatty acids with polyhydroxyethylene sorbitans such as polyethyleneglycol sorbitan monolaurate, monostearate or monooleate, ethers of fatty alcohols with polyhydroxyethylene, esters of fatty acids with polyhydroxyethylene, copolymers of ethylene oxide and propylene oxide and ethoxylated triglycerides.

13. The orally disintegrating tablet according to claim 13, wherein the solubilizer is polysorbate 80 present in the range from about 0.02% w/w to about 6% w/w, preferably in the range from about 0.05% w/w to about 4% w/w.

14. The orally disintegrating tablet according to claim 6, wherein the stabilizing agent is selected from the group consisting of calcium carbonate, calcium hydroxide, magnesium carbonate, magnesium hydroxide, magnesium silicate, magnesium aluminate, or aluminum magnesium hydroxide and their combinations thereof.

15. The orally disintegrating tablet according to claim 15, wherein the stabilizing agent is calcium carbonate present in the range from about 2% w/w to about 15% w/w, preferably in the range of about 4% w/w to about 9% w/w.

16. The orally disintegrating tablet according to claim 6, wherein the sweetener is selected from the group consisting of cyclamate, acesulfame potassium, neo hesperidin dihydrochalcone, monoammonium glycyrrhizinate, saccharin sodium, sucralose, saccharin, aspartame, and combinations thereof.

17. The orally disintegrating tablet according to claim 6, wherein the flavoring agent is selected from menthol, floral fennel flavor, mint powder, vanillin, or orange flavor.

18. The orally disintegrating tablet according to claim 1, further comprises magnesium stearate, hydroxypropyl cellulose, calcium carbonate, polysorbate 80, aspartame, and orange flavor.

19. The orally disintegrating tablet according to claim 1, wherein the D 90 particle size of atorvastatin or a pharmaceutically acceptable salt thereof, is in the range of about 20 to 200 μm, more preferably in the range of about 50 to 150 μm.

20. The orally disintegrating tablet according to claim 1, wherein the D 50 particle size of atorvastatin or a pharmaceutically acceptable salt thereof, is in the range of about 1 to 20 μm, more preferably in the range of about 4 to 10 μm.

21. The orally disintegrating tablet according to claim 1, wherein the D 10 particle size of atorvastatin or a pharmaceutically acceptable salt thereof, is in the range of about 0.25 to 15 μm, more preferably in the range of about 0.5 to 10 μm.

22. The orally disintegrating tablet according to claim 1, wherein the orally disintegrating tablet is manufactured by the wet granulation method comprising step of:
(a) Sieving atorvastatin calcium or pharmaceutically acceptable salts thereof, microcrystalline cellulose 101, hydroxyl propyl cellulose, mannitol, croscarmellose sodium, and calcium carbonate are sieved separately through a 40# sieve, and aspartame, orange flavor, and magnesium stearate separately through a 60# sieve;
(b) Preparing binder solution by adding one half quantity of hydroxy propyl cellulose and polysorbate 80 to a sufficient quantity of purified water;
(c) Mixing of previously sifted atorvastatin calcium and microcrystalline cellulose 101 into the rapid mixer granulator and mix for 10 minutes by keeping the impeller slow and the chopper off;
(d) Adding binder solution slowly to the dry mix with continuous mixing in a rapid mixer granulator for 5 min by keeping the impeller slow and the chopper off and then granulating the mixture for a further 2 min;
(e) Drying the above granulated blend in a dryer at 50° C.±5° C. till the loss on drying of the dried granules becomes not more than 2.5%;
(f) passing the dried granules through a 24# sieve;
(g) Mixing previously shifted mannitol, remaining quantity of hydroxyl propyl cellulose, croscarmellose sodium, calcium carbonate, aspartame, and orange flavor to the dried granules prepared in step (f) and blended for 15 minutes in a blender;
(h) Adding magnesium stearate to the pre-lubricated blend of step (g) and mix properly for 5 minutes; and
(i) Compressing the resulting mixture into the tablet dosage form.

23. The orally disintegrating tablet according to claim 1, wherein the orally disintegrating tablet is disintegrated in the buccal cavity upon contact with saliva in less than 3 minutes, preferably less than 90 seconds.

24. The orally disintegrating tablet according to claim 1, wherein 80% of the atorvastatin or pharmaceutically acceptable salts thereof, is released within 60 minutes, preferably more than 90% is released within 50 minutes.

25. The orally disintegrating tablet according to claim 1, is for the treatment or prevention of dyslipidemias, hypercholesterolemia, dysbetalipoproteinemia, myocardial infarction, stroke, revascularization procedures, hospitalization for congestive heart failure and angina in patients with coronary heart disease.
